(19) United States
(12) Patent Application Publication
(10) Pub. No.: US 2005/0181490 A1
(43) Pub. Date: Aug. 18, 2005

(54) Fermentation process for preparing coenzyme Q10 by the recombinant Agrobacterium tumefaciens

(76) Inventors: Soo-Ryun Cheong, Seoul (KR); Sang-Young Kim, Gwangmyeong-Shi (KR); Jung-Kul Lee, Seoul (KR); Hyeon-Cheol Lee, Seoul (KR); Suk-Jin Ha, Suwon-Shi (KR); Bong-Seoung Koo, Seoul (KR); Ji-Hyun Yoo, Seoul (KR)

Correspondence Address: KENYON & KENYON, 1500 K STREET NW, SUITE 700, WASHINGTON, DC, 20005, US

(21) Appl. No.: 11/042,209

(22) Filed: Jan. 26, 2005

(30) Foreign Application Priority Data
Feb. 16, 2004 (KR) 2004-9921

(51) Int. Cl. C12P 7/66; C12N 9/12; C12N 15/74; C12N 1/21

(52) U.S. Cl. 435/133; 435/471; 435/252.2; 435/194

(57) ABSTRACT

The present invention relates to a transformed Agrabacterium tumefaciens BNQ-pGPRX11 (Accession No. KCCM-10554) harboring a recombinant expression vector (pGPRX11). Further, the present invention also provides a fermentation method for maximum production of coenzyme Q₁₀ using a transformed Agrabacterium tumefaciens deposited to Korean Culture Center of Microorganism with accession number KCCM-10554 comprising the steps of: i) fermenting transformed cells on production medium comprising 30˜50 g/L of corn steep powder, 0.3˜0.7 g/L of KH₂PO₄, 0.3˜0.7 g/L of K₂HPO₄, 12˜18 g/L of ammonium sulfate, 1.5˜2.5 g/L of lactic acid, 0.2˜0.3 g/L of magnesium sulfate on condition that aeration rate of the medium is 0.8˜1.2 volume of air per volume of medium per minute, temperature 30˜34° C. and pH is 6.0˜8.0; ii) removing the transformed cells and other residue from the fermentation medium; and iii) separating and recovering coenzyme Q₁₀ from the fermentation medium of step (ii).

### BACKGROUND OF THE INVENTION

The present invention relates to a transformed microorganism strain producing coenzyme Q₁₀ in the high productivity and to a process for preparing coenzyme Q₁₀ using the transformed microorganism strain belonged to Agrabacterium tumefaciens species.

In particular, the present invention concerns to the construction of DXS and DPS gene expression vector pGPRX11 and its transformed strain Agrabacterium tumefaciens BNQ (KCCM-10554) harboring said expression vector producing coenzyme Q₁₀. Also, it is relates to a process for preparing coenzyme Q₁₀ in an aerobic condition using said recombinant.

Coenzyme Q₁₀ (2,3-dimethoxy-5-methyl-6-decaprenyl-1,4-benzoquinone) was firstly found as the component of bovine heart mitochondria by Crane et al., in 1957. The following chemical structure of coenzyme Q₁₀ has been disclosed since 1958 by Folkers et. al.

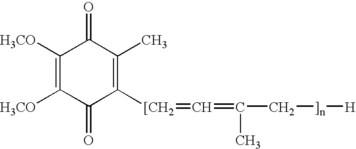

It is a kind of lipid-soluble quinone (also called ‘ubiquinone’) having similar properties as vitamins and it has been known as an essential material for maintaining healthy status of organisms. As a transporter of electrons and protons essential to the survival of organism, coenzyme Q₁₀ plays various roles for sustaining ATP synthesis within mitochondria inner membrane; for maintaining cell skeleton and metabolism by stabilizing cell membranes. It also acts as an anti-oxidant against reactive oxygen species to prevent oxidative damages to DNA, lipids, proteins and the like. It also functions to prevent or alleviate the symptoms of cardiovascular diseases, tumor diseases, neuro-pathogenic diseases and the like.

For producing coenzyme Q₁₀, 3 different kind of preparation methods have been developed, which are i) extraction method from animal or plant tissues, ii) chemical synthesis method and iii) a fermentation method using microorganism. Among them, fermentation process using microorganism has been regarded as commercially available and safe process for producing coenzyme Q₁₀.

It has been reported that coenzyme Q₁₀ has been produced by microorganisms, such as, Cryptococcus laurentii FERM-P4834, Rhodotorula glutinis FERM-P4835, Sporobolomyces salmonicolor FERM-4836, Trichosporon sp. FERM-P4650, Aureobasidium sp. and the like.

The commercially marketed coenzyme Q₁₀ have been produced from many companies, such as, Kyowa Co., Ltd., Nisshin Flourmilling Co., Ltd., Kaneta, Ajinomoto and Merck using biological process extracted from microorganism cell. However, the products manufactured from these companies showed low productivity and high cost, because concentration of coenzyme Q₁₀ in cells are too low to extract it in a commercial scale.

For the biosynthesis of coenzyme Q₁₀ in microorganisms, a complicated multi-step pathway is required, where many enzymes are involved. In general, however, it is considered that three major steps are involved, which are, i) synthesizing step for decaprenyl diphosphate of side chain portion of coenzyme Q₁₀, ii) cyclization forming step for quinone ring, and iii) completing step for coenzyme Q₁₀ by combining these two compounds and sequentially transforming their constituents.

The most important step among them has been considered as DPS (decaprenyl diphosphate) forming step which consists of the side chain of coenzyme Q₁₀. Further, DXS (1-deoxy-D-xylulose 5-phosphate synthase) is also involved in preparing isopentenyl diphosphate of side chain constituent. Therefore, in order to enhance the productivity of coenzyme Q₁₀, the introduction of these two genes expressing DPS and DXS to host cell has been required.

Although DPS isolated from a few microorganisms such as Schizosaccharomyces pombe, Gluconobacter suboxydans, etc. has been tried to be introduced into E. coli, satisfactory productivity of coenzyme Q₁₀ has not been accomplished in these recombinant bacteria. In addition, even though DXS is introduced into E. coli for the production of coenzyme Q₁₀, the productivity of coenzyme Q₁₀ is still unsatisfactory. Therefore, E. coli is seldom used as microorganism for coenzyme Q₁₀, even though it has been reported that productivity of coenzyme Q₈ by E. coli can be improved when DXS is overexpressed in E. coli.

Therefore, it is necessary to isolate the microorganism strain over-expressing the key enzymes for producing a large amount of coenzyme Q₁₀. Further, it is also required to fix the fermentation conditions for maximum production of both biomass containing coenzyme Q₁₀ and contents of coenzyme Q₁₀ in biomass by controlling the fermentation temperature, fermentation pH, aeration condition, stirring condition and dissolved oxygen content in an industrial scale.

### SUMMARY OF THE INVENTION

The object of invention is to provide an isolated 1-deoxy-D-xylulose 5-phosphate synthase (DXS) gene of SEQ ID NO: 1 from Agrobacterium tumefaciens.

Another object of invention is to provide a 1-deoxy-D-xylulose 5-phosphate synthase (DXS) of SEQ ID NO: 2.

The further object of invention is to provide a recombinant expression vector (pGPRX11) inserted with both decaprenyl diphosphate (DPS) gene and 1-deoxy-D-xylulose 5-phosphate synthase (DXS) gene.

The further object of invention is to provide a transformed Agrabacterium tumefaciens BNQ-pGPRX11 (Accession No. KCCM-10554) by a recombinant expression vector (pGPRX11).

The further object of invention is to provide a fermentation method for maximum production of coenzyme Q₁₀ using a transformed Agrobacterium tumefaciens deposited to Korean Culture Center of Microorganism with accession number KCCM-10554 comprising the steps of: i) fermenting transformed cells on production medium comprising 30˜50 g/L of corn steep powder, 0.3˜0.7 g/L of KH₂PO₄, 0.3˜0.7 g/L of K₂HPO₄, 12˜18 g/L of ammonium sulfate, 1.5˜2.5 g/L of lactic acid, 0.2˜0.3 g/L of magnesium sulfate on condition that aeration rate of the medium is 0.8˜1.2 volume of air per volume of medium per minute, temperature 30˜34° C. and pH is 6.0˜8.0; ii) removing the transformed cells and other residue from the fermentation medium; and iii) separating and recovering coenzyme Q₁₀ from the fermentation medium of step (ii).

Further, the fermentation process is carried out by pH-stat fed batch culture and the content of dissolved oxygen is controlled at 0.01˜10%.

### BRIEF DESCRIPTION OF THE DRAWINGS

FIG. 1 shows the 16S ribosomal RNA partial sequence of Agrobacterium tumefaciens BNQ producing coenzyme Q₁₀ of the present invention.

FIG. 2 shows the entire nucleotide sequence and amino acid sequence of 1-deoxy-D-xylulose 5-phosphate synthase (DXS) cloned from A. tumefaciens. The size of enzyme is 68 kDa.

FIG. 3 shows the amino acids homology among the amino acid sequences of cloned DXS of present invention and other DXS sequences derived from other microorganisms, after alignment for the comparison. Asterisk indicates the identical places.

A conserved motif having histidine residues considered to be associated with hydrogen transfer is indicated in the box, and a region predicted to be a binding site of thiamine diphosphate is shadowed. Followings are microorganisms from which each DXS was derived. ATUM: DXS cloned by the inventors, ECLI: E. coli, HINF: H. influenzae, BSUB: B. subtilis, RCAP: R. capsulatus, SYNE: Synechocystis sp. PCC6803, ATHA: A. thaliana, and CLA190: Streptomyces sp. strain CL190.

FIG. 4 shows the structure of recombinant plasmid pQX11.

FIG. 5 shows the SDS-PAGE analysis of DXS expressed from E coli harboring pQX11.

lane A: wild type E. coli, lane B: E. coli transformed with pQX11 with treatment of IPTG, lane C: The purified standard of expressed DXS, lane D: a marker.

FIG. 6 shows the chromatogram showing the occurrence of DXS enzyme which is expressed from E. coli. A: The result using a cell extract of wild type E. coli, B: The result using purified DXS, C: The result using the same condition as B without addition of thiamin diphosphate.

FIG. 7 shows the structure of recombinant plasmids pGP85 and pGX22.

FIG. 8 shows the structure of recombinant plasmid pGPRX11.

FIG. 9 shows the production of coenzyme Q₁₀ and bacterial cell growth according to the lapse of fermentation time using pH-stat by fed-batch culture in 5 L fermenter.

### DETAILED DESCRIPTION OF THE INVENTION

The present invention consists of two parts; i) the construction of transformed strain, and ii) the optimization of fermentation conditions.

In order to accomplish the construction of transformed strain, following steps are required; i) cloning the DXS gene from A. tumefaciens; ii) establishment of an expression system of DXS in E. coli; iii) expression of DXS by IPTG induction and activity confirmation of expressed DXS; iv) construction of recombinant expression vector pGPRX11 containing DPS and DXS genes; and v) construction of recombinant strain of A. tumefaciens BNQ-pGPRX11 harboring said recombinant vector.

In order to optimize the fermentation conditions, we have searched the optimal conditions for recombinant strain of A. tumefaciens BNQ-pGPRX11, for example temperature, pH, agitation condition, aeration condition, etc. To achieve the most suitable conditions for production of high concentration of coenzyme Q₁₀ and for the increased biomass quantity, control of dissolved oxygen and fed-batch culture are required. Further, for increased production of coenzyme Q₁₀, the selection of medium in an industrial scale, for example, medium comprising corn steep solid, ammonium sulfate, potassium phosphate monobasic, potassium diphosphate, magnesium sulfate, lactic acid, etc. is also required.

To obtain a DXS gene, A. tumefaciens strains producing coenzyme Q₁₀ is employed. Then, the entire DXS gene of A. tumefaciens is cloned on the basis of previously known DXS gene sequences from other microorganism with the supposition that size of gene may be about 1.9 kb. For cloning DXS gene, E. coli XL1-Blue and cloning vector pSTBlue-1 are used. Further, E. coli JM109 and expression vector pQE30 (QIAGEN) are also used for expression of DXS in E. coli. Both E. coli and A. tumefaciens have been cultured in LB medium as well as LB agar plate. E. coli cultivation is carried out under the conditions of 220 rpm at 37° C. for 12 hours, whereas A. tumefaciens cultivation is carried out under the condition of 240 rpm at 30° C. for 16˜24 hours.

To obtain the DXS gene to be integrated with A. tumefaciens, DNA fragments amplified by PCR using pQX22 as a template are firstly collected. Then, to obtain the DPS gene, DNA fragments amplified by PCR using pQD22 (Biotechnol. Progress 2003) as a template are subsequently collected. Then, obtained DNA fragments are cloned into the pST1-Blue vector. Finally, these fragments are cloned into pGA748, an expression vector for A. tumefaciens.

After recombinant vectors pGP85 harboring DPS and pGX22 harboring DXS are firstly transformed into E. coli to secure a large amount of plasmids, DNA sequencing is measured. Then, completed recombinant vectors pGP85 and pGX22 are infused into a coenzyme Q₁₀-producing strain by electroporation method. Finally, transformed strain is selected in the LB selection medium containing 3 μg/ml of tetracycline. The selected transformed strain infused with DPS is designated as BNQ-pGP85, while the transformed strain infused with DXS is designated as BNQ-pGX22.

On the other hand, in order to construct a plasmid capable of expressing DPS and DXS simultaneously, DXS having ribosomal binding site (RBS) is obtained by PCR. Then, obtained PCR product is inserted into plasmid pGP85. Obtained plasmid containing both DPS and DXS is designated as pGPRX11. The transformed coenzyme Q₁₀-producing strain is confirmed by colony PCR with a pair of primers comprising internal DNA sequences.

Finally, the transformed strain has been deposited as A. tumefaciens BNQ-pGPRX11 in the Korea Culture Center of Microorganism located at 361-221, Yurim building, Hongje 1-Dong, Seodaemun-Gu, Seoul, Korea on Jan. 2, 2004 with the accession number KCCM-10554 under the Budapest Treaty.

Most of DNA are confirmed by agarose gel electophoresis (TAE buffer 1%) and the purification of DNA band is carried out by Geneclean II gel extractor (Q-biogene, USA). Ligation of DNA is carried out by T4 DNA ligase (Boehringer Mannheim).

The composition of growth medium for cultivation of transformed strain is set forth on Table 1 and the composition of production medium for mass production of coenzyme Q₁₀ is also set forth on Table 2.

TABLE 1
Composition of growth medium

| Component | Concentration(g/L) |
|---|---|
| Sucrose | 50 |
| Yeast extract | 15 |
| Peptone | 15 |
| NaCl | 7.5 |

TABLE 2
Composition of production medium

| Component | Concentration(g/L) |
|---|---|
| Corn steep solid | 40 |
| Ammonium sulfate | 10 |
| KH₂PO₄ | 0.5 |
| K₂HPO₄ | 0.5 |
| MgSO₄.7H₂O | 0.25 |
| Sucrose | 50 |

The cultivation in growth medium is carried out by following procedure; i) inoculating the strain to the 100 ml of growth medium in the 500 ml triangle flask, ii) agitating and cultivating the strain under the conditions of 200 rpm at 32° C. for 16˜24 hours. Further, the main culture is also carried out in a 5 L fermenter (KoBiotech) for researching the optimization culture conditions. Then, the main culture is carried out in various conditions by varying the temperature (25° C.˜35° C.), pH(6.0˜8.0), agitation condition (300˜600 rpm) and aeration condition (0.5˜2.0 vvm) for about 4 days.

To enhance the biomass quantity, method for controlling dissolved oxygen and fed-batch culture are adopted. Dissolved oxygen amount is adjusted in the range of 0˜30% by varying agitation speed for determining the optimal cultivation. Further, fed-batch culture is also employed by intermittently adding carbon source to the medium to enhance the biomass quantity. Fed-batch culture using pH-stat is also preferred.

Optimal medium selection is carried out for maximum production of coenzyme Q₁₀ in biomass., Further, optimal concentrations of each medium composition, such as, corn steep powder, ammonium sulfate, potassium phosphate monobasic, potassium diphosphate, magnesium sulfate, lactic acid, etc. are also established.

The present invention can be explained by following examples. However, the scope of present invention shall not be limited by following examples.

### EXAMPLE 1

### Separation and Identification of A. tumefaciens Strain

Preferred strains producing coenzyme Q₁₀ were primarily screened from approximately 1×10⁶ bacteria obtained on LB solid media from the soil samples. Then, secondary screening from them can separate about 500 bacteria considered as high growth rate of biomass and high productivity of coenzyme Q₁₀. Finally the bacterium to be highest in productivity of coenzyme Q₁₀ was screened. Identification of said bacterium finally screened to produce coenzyme Q₁₀ at high concentration was carried out by 16S rDNA sequencing (Jukes, T. H. & Cantor, C. R. 1969).

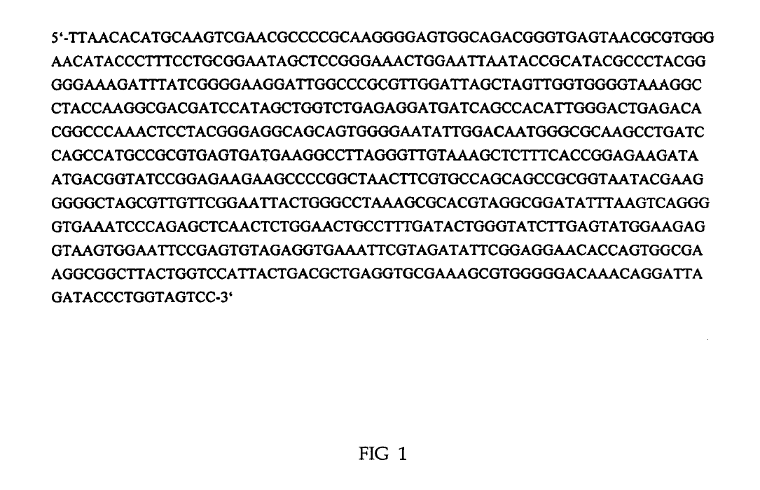

FIG. 1 shows the 16S ribosomal RNA partial sequence of Agrobacterium tumefaciens BNQ producing coenzyme Q₁₀ of the present invention. Further, the analysis results of homology among 16s rRNA sequence from analog species are shown in Table 3.

TABLE 3
The homology among 16s rRNA sequence from analog species for
producing coenzyme Q₁₀

| | Accession | |
|---|---|---|
| Strain | No. | % Similarity |
| Agrobacterium tumefaciens NCPPB 2437T | D14500 | 100.00 |
| Agrobacterium rubi IFO 13261T | D14503 | 98.28 |
| Agrobacterium larrymoorei ATCC 51759T | Z30542 | 98.00 |
| Rhizobium huautlense S02T | AF025852 | 97.43 |
| Rhizobium galegae ATCC 43677T | D11343 | 96.84 |
| Rhizobium mongolense USDA 1844T | U89817 | 96.147 |
| Agrobacterium vitis NCPPB 3554T | D14502 | 95.83 |
| Rhizobium leguminosarum IAM 12609T | D14513 | 95.40 |
| Rhizobium hainanense CCBAU 57015T | U71078 | 95.29 |
| Rhizobium etli CFN 42T | U28916 | 95.14 |
| Agrobacterium rhizogenes IFO 13257T | D14501 | 94.97 |
| Rhizobium tropici IFO 15247T | D11344 | 93.98 |
| Bradyrhizobium japonicum USDA 6T | U69638 | 88.43 |

In above homology analysis of the 16s rDNA partial sequence of strains producing coenzyme Q₁₀ at high concentrations, the selected strain in example 1 was identified as A. tumefaciens strain and designated as A. tumefaciens BNQ.

### EXAMPLE 2

### Cloning of A. tumefaciens DXS Gene

For cloning the DXS gene, cDNA of A. tumefaciens was separated. A pair of PCR primers were manufactured referring to closest known DXS amino acid sequences from other strain. Followings are a pair of primers for cloning the DXS gene from A. tumefaciens.

| | |
|---|---|
| F1 | |
| 5′-CAAAATCCTCCTACCGGCCGC-3 | (SEQ ID NO: 3) |
| R1 | |
| 5′-CGCTGCTGTCGCGATGCC-3′ | (SEQ ID NO: 4) |

The above primers were used to amplify 873 bp of DNA from cDNA of A. tumefaciens. From the comparison with DNA sequences of DXS derived from various microorganisms, it was found that the obtained PCR products has the highest similarity with the existing DXS. In order to obtain the entire DXS gene, 5′- and 3′-RACE (rapid amplification of cDNA ends) methods were employed, which were carried out according to the manufacturer's manual (Roche Diagnostics GmbH, Manheim, Germany) using 5′- and 3′-RACE kit. Primers specific for DXS genes were manufactured for each RACE.

| | |
|---|---|
| i) Primers for 5′-RACE | |
| SP1 | |
| 5′-CTCGGCCATCTTGTCGAGGCC-3′ | (SEQ ID NO: 5) |
| SP2 | |
| 5′-ATTCGGCATGGCGGCGGTGAC-3′ | (SEQ ID NO: 6) |
| SP3 | |
| 5′-GCCGACGATCTTGTCGTCGAG-3′ | (SEQ ID NO: 7) |
| ii) Primer for 3′-RACE | |
| SP4 | |
| 5′-GCAGCTTTCGGTCGCCAAG-3′ | (SEQ ID NO: 8) |

Effectuation of RACE using these primers amplified cDNA containing DXS. In order to obtain an open reading frame of DXS, PCR primers beginning by start codon and ending by termination codon were prepared. A BamHI restriction site was included in a forward primer and a HindIII restriction site was also included in a reverse primer to facilitate cloning procedure. DNA sequences for each primer are as follows.

| | |
|---|---|
| DXF1 | |
| 5′-GGATCCTTGACCGGAATGCCACAGAC-3′ | (SEQ ID NO: 9) |
| DXB1 | |
| 5′-AAGCTTCTCAGCCGGCGAAACCGAC-3′ | (SEQ ID NO: 10) |

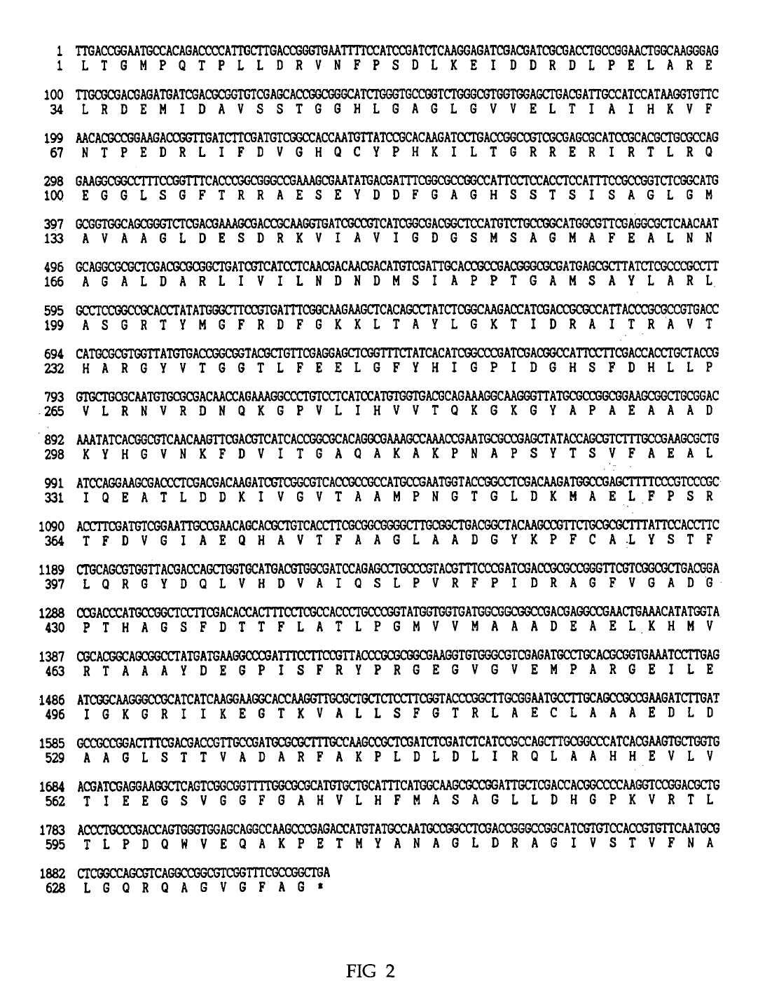

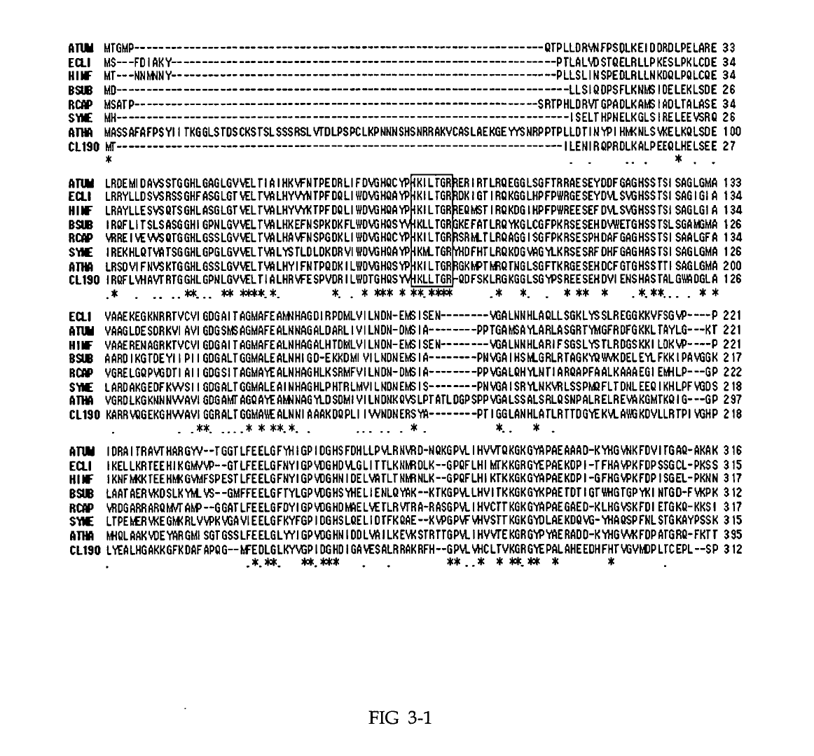

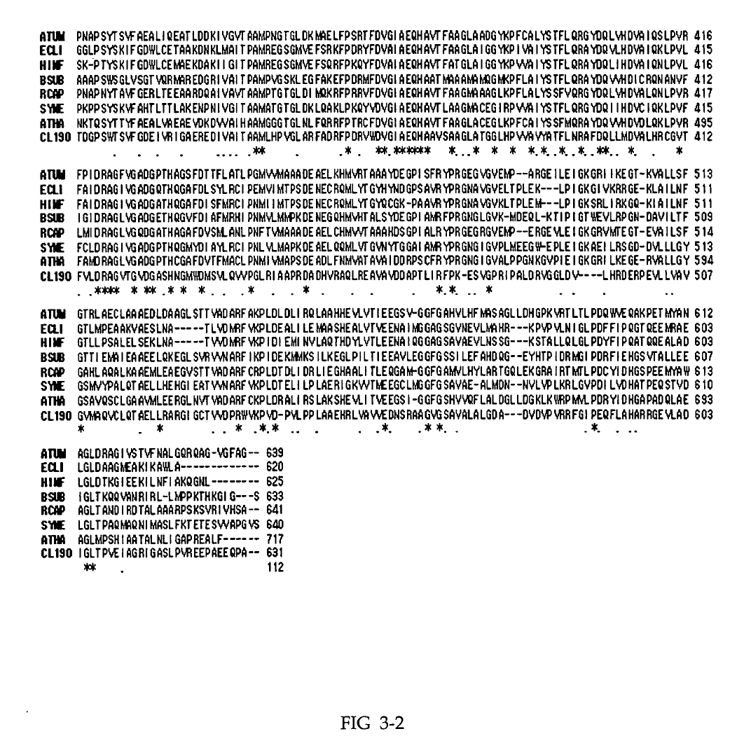

PCR using these primers resulted in 1,920 bp of DXS cDNA flanked with BamHI and HindIII restriction sites in 5′ and 3′ position respectively (FIG. 2). The obtained cDNA was translated and it was also compared with previously known amino acid sequences of DXS. The result showed 37˜59% similarity compared to previously known sequences and it was confirmed that thiamine diphosphate binding domain, which is an element essentially found in amino acid sequence of DXS, and that histidine residues considered to be associated with hydrogen transfer were well conserved (FIG. 3).

### EXAMPLE 3

### Establishment of Expression System in E. coli of DXS Derived from A. tumefaciens

In order to determine the activity of DXS, this enzyme was expressed in E. coli, after cloning from A. tumefaciens. A pQE system (QIAGEN, USA) well known among E. coli recombinant protein expression system was used, because its system contained T5 promoter.

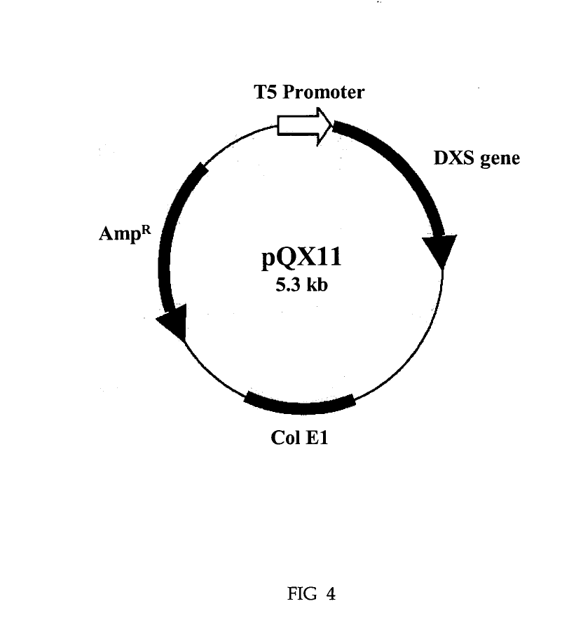

Because of DXS gene fragment including a BamHI restriction site at 5′ end and a HindIII restriction site at 3′ end, both restriction enzymes BamHI and HindIII were simultaneously treated. After extraction on agarose gel, a 1.9 kb DXS gene was separated and purified. Then, such BamHI and HindIII double restriction enzyme treatment was also performed in expression vector pQE30 (3.4 kb). Consequently, 1.9 kb of DXS gene was cloned and inserted into the vector, which was designated as pQX11 (FIG. 4).

### EXAMPLE 4

### Expression and Purification of DXS Gene in E. coli through IPTG Induction

E. coli JM109 transformed with pQX11 vector was incubated and it was subsequently treated with 0.1 mM of IPTG at 30° C. for 2 hours when optical density (600 nm) is 0.5. Then, the expression of DXS was induced. After soluble fractions of expressed proteins were mixed with Ni-NTA resin, the mixture was passed through the column. The active site part was exclusively separated with a buffer containing 240 mM of imidazole. The expressed proteins of interest were isolated using 10% SDS electrophoresis. The test material was mixed and boiled with sample solution (1% SDS, 5% β-mercaptoethanol, 10% glycerol, bromophenolblue). The dye, Coomassie Brilliant Blue R-250, was also used for detection.

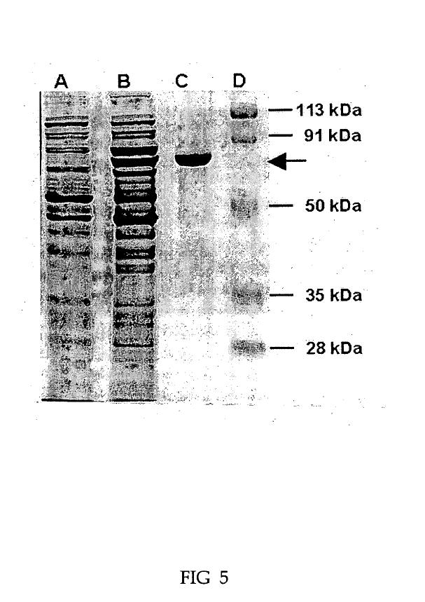

SDS-PAGE data was shown in FIG. 5 using pQE expression system. Based on amino acid sequence data derived from DNA sequencing, the dimension of DXS was estimated to be 68.05 kDa, which was confirmed by the band in SDS-PAGE.

### EXAMPLE 5

### Measurement of DXS Activity

In order to measure DXS activity, 20 μg of purified DXS was mixed with 40 mM Tris-HCl buffer, pH 8.0 containing 1 mM magnesium chloride, 1 mM thiamine diphosphate, 1 mM pyruvate, 2 mM glyceraldehyde 3-phosphate and 5 mM mercaptoethanol. Then, the mixture was reacted at 37° C. for 1 hour. After centrifuging the reacted mixture with 13,000 rpm, supernatant was collected. Then, the reaction product was analyzed by HPLC using Zorbax-NH₂ column (Agilent technologies, Palo Alto, Calif.) having 195 nm ultraviolet detector. The eluant was a 100 mM of potassium phosphate monobasic solution, pH 3.5, and flow rate was 1.3 ml/min.

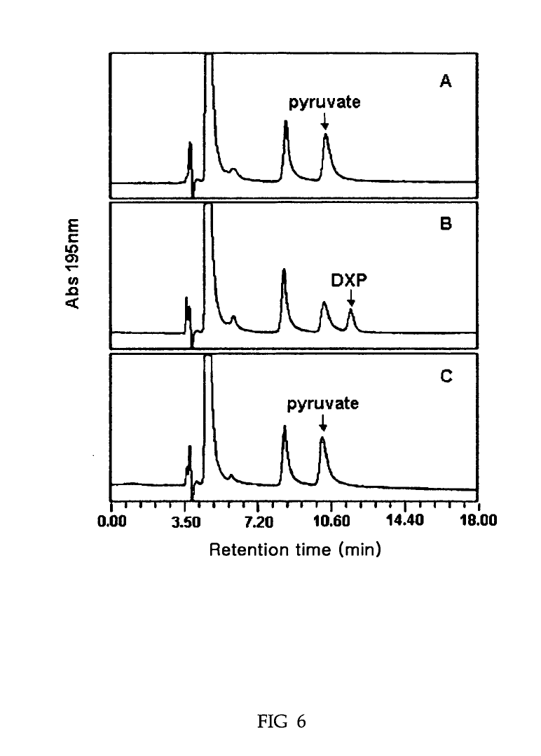

It was confirmed by HPLC chromatography that DXP (1-deoxy-D-xylulose-5-phosphate) was formed as expected through the analysis of enzyme reaction product (FIG. 6). Further, since DXP was not produced in enzyme reaction without TDP (thiamine diphosphate), the inventors confirmed that a gene cloned from A. tumefaciens is DXS.

### EXAMPLE 6

### Construction of Recombinant Plasmids

In order to construct cDNA comprising genes of DPS and DXS, PCR was carried out with recombinant plasmids pQD22 and pQX11 as templates, which had been previously developed by the inventors. A pair of primer sequences for amplifying cDNA of DPS are as follows. The 5′ DNA fragment of DPS has HindIII restriction site, while the 3′ DNA fragment of DPS has MluI restriction site.

DFF8 (SEQ ID NO: 11)
5′-AAGCTTTTGCCGCGCAAGGCGTCAG-3′
DFB5 (SEQ ID NO: 12)
5′-ACGCGTTCAGTTGAGACGCTCGATGCA G-3′

A pair of primer sequences for amplifying cDNA of DXS are as follows. The 5′ DNA fragment of DXS has HindIII restriction site, while the 3′ DNA fragment of DPS has EcoRI restriction site.

| | |
|---|---|
| DXF2 | |
| 5′-AAGCTTTTGACCGGAATGCCACAGAC-3′ | (SEQ ID NO: 13) |
| DXB2 | |
| 5′-GAATTCTCAGCCGGCGAAACCGAC-3′ | (SEQ ID NO: 14) |

PCR products were developed in agarose gel electrophoresis and obtained band was purified. Then, purified DNA fragments was ligated with cloning vector pSTBlue-1(Novagen Co.). Recombinant plasmid was inserted into E. coli XL1-Blue and it was cultivated in 50 mg/L ampicillin medium overnight.

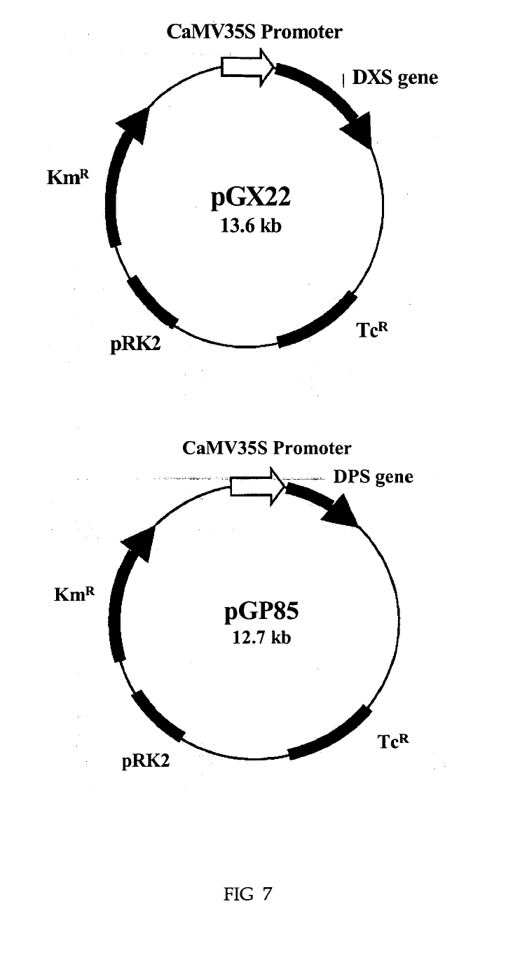

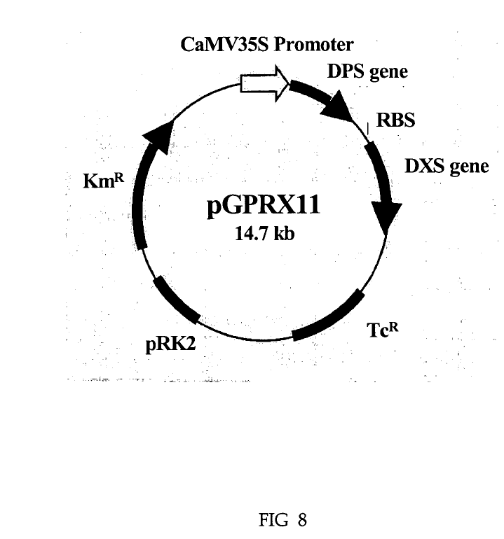

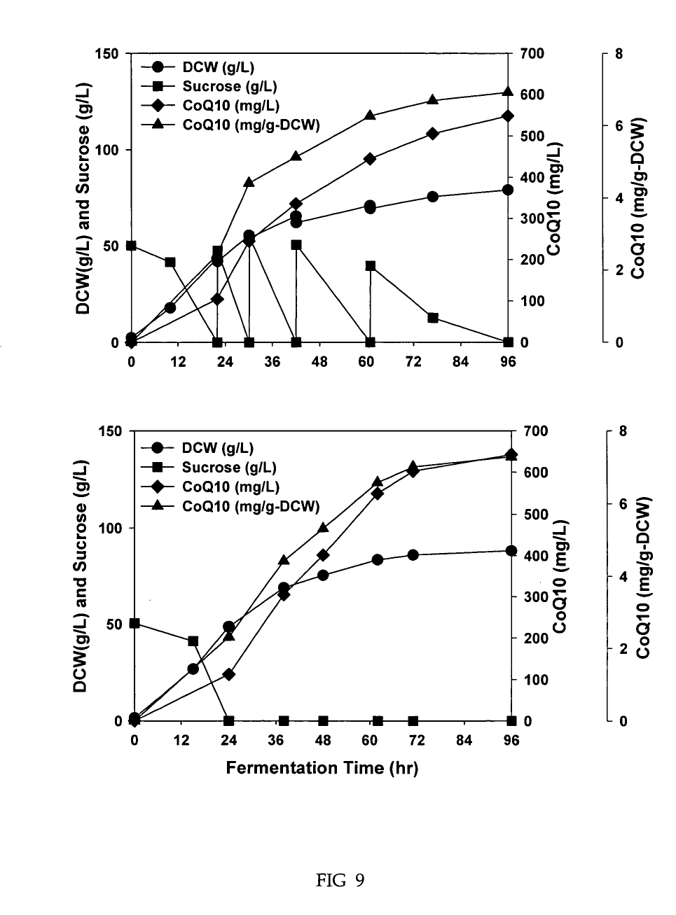

Insert DNA in recombinant plasmid was confirmed by analysis of DNA sequence with confirmation of restriction map. cDNA fragment coding DPS was obtained by restriction enzyme HindIII and MluI and cDNA fragment coding DXS was obtained by restriction enzyme HindIII and EcoRI. Each cDNA segment was ligated to expression vector pGA748 for A. tumefaciens. Then, E. coli was transformed by expression vector. Each of the resulting plasmids was designated as pGP85 and pGX22 (FIG. 7).

In order to construct an expression vector capable to express DPS and DXS concurrently, PCR was carried out with RBS-containing DXS plasmid pGX22 as a template. The 5′ DNA fragment of DXS has XhoI restriction site, while the 3′ DNA fragment of DXS has ClaI restriction site.

pGPXF1 (SEQ ID NO: 15)
5′-CTCGAGGAAGTTCATTTCATTTGGAGAGG-3′
pGPXB1 (SEQ ID NO: 16)
5′-ATCGATTCAGCCGGCGAAACCGAC-3′

PCR product was digested with restriction enzymes XhoI and ClaI, and it was clearly eluted after electrophoresis on agarose gel. After DXS fragment was ligated to plasmid pGP85, E. coli was transformed. The plasmid extracted and sequenced from the transformed E. coli was designated as pGPRX11.

### EXAMPLE 7

### Preparation of Recombinant Bacteria Using Electroporation

To obtain competent cells, coenzyme Q₁₀-producing bacterium BNQ605 was cultivated in LB medium until the cell density became 5˜10×10⁷ cell/ml. After centrifuge, obtained cells were washed with EPB1 buffer (20 mM Hepes pH 7.2, 5% glycerol) 3 times and they were suspended with EPB2 buffer (5 mM Hepes pH 7.2, 15% glycerol) The cells were stored at −70° C.

7˜10 μg of recombinant plasmid pGP85, pGX22 and pGPRX11 were inserted to 80 μl of competent cell obtained above using electroporator (MicroPulser, BIORAD) by electric stimulation of 25 μF, 2.5 kV for 0.5 second. After the addition of 1 ml of LB broth and incubation at 30° C. for 2˜3 hours, these cells were plated into LB solid medium supplemented with 3 μg/ml of tetracycline. Then, they were incubated at 30° C. for 72 hours. Finally, cell line colonies to be inserted with DNA of interest was screened.

Insertion of recombinant plasmid was confirmed by colony PCR. A pair of primers used for colony PCR was based on the pre- and post-DNA sequences of multi-cloning site (MCS) of expression vector pGA748. Followings are primer sequences.

| | |
|---|---|
| p748F1 | |
| 5′-ATCCTTCGCAAGACCCTTC-3′ | (SEQ ID NO: 17) |
| p748B1 | |
| 5′-GCTTAGCTCATCGCAGATC-3′ | (SEQ ID NO: 18) |

The consequence of Colony PCR confirmed that recombinant expression vectors pGP85 and pGX22 had been normally inserted into a coenzyme Q₁₀-producing strain of A. tumefaciens BNQ 0605. Among transformed strains, the strain transformed with pGP85 was designated as BNQ-pGP85; the strain transformed with pGX22 was designated as BNQ-pGX22; and the strain transformed with pGPRX11 was designated as BNQ-pGPRX11 (Accession Number KCCM-10554).

### EXAMPLE 8

### Determination of Coenzyme Q₁₀ Productivity

In order to determine coenzyme Q₁₀ productivity of recombinant strains BNQ-pGP85, BNQ-pGX22 and BNQ-pGPRX11 prepared in example 6, these strains were inoculated and cultured in 5 ml of LB broth medium containing 3 μg/ml of tetracycline at 30° C. 240 rpm overnight. As a control, normal strain BNQ-pGA748 in which only plasmid pGA748 was inserted was used and cultured in the same conditions described as above. The results of growth of coenzyme Q₁₀ producing strains and the results of coenzyme Q₁₀ productivity are listed on Table 4 below.

TABLE 4
Comparison the growth of recombinant strains and their coenzyme Q₁₀
productivity

| | Growth | CoQ₁₀ |
|---|---|---|
| Strain | (OD₆₆₀) | (μg/g-DCW) |
| BNQ-pGA748 | 2.42 | 445 |
| BNQ-pGX22 | 2.86 | 561 |
| BNQ-pGP85 | 2.73 | 585 |
| BNQ-pGPRX11 | 2.15 | 909 |

### EXAMPLE 9

### Optimization of a Basic Culture Condition

The above identified recombinant strain BNQ-pGPRX11 was used to perform the optimization experiment under the basic culture condition. By effectuating incubation varying the conditions, such as, temperature (25° C.˜35° C.), pH (6.0˜8.0), agitation condition (300˜600 rpm), aeration condition (0.5˜2.0 vvm), it was found that 32° C. of optimum temperature, 7.0 of optimum pH, 500 rpm of agitation condition and 1.0 vvm of aeration condition were confirmed to be an optimal condition suitable for growth of biomass and biosynthesis of coenzyme Q₁₀. Table 5 shows the comparison of cell broth, amount of coenzyme Q₁₀ according to the cultivation of strain BNQ-pGPRX11.

TABLE 5
Cultivation conditions of BNQ-pGPRX11 for producing coenzyme Q₁₀

| | | Cell mass | CoQ₁₀ | CoQ₁₀ |
|---|---|---|---|---|
| | | (g/L) | (mg/L) | (mg/g-DCW) |
| Temp. | 25 | 34.3 | 130.1 | 3.79 |
| (° C.) | 30 | 37.4 | 188.4 | 5.04 |
| | 32 | 38.4 | 203.2 | 5.29 |
| | 35 | 39.1 | 172.3 | 4.41 |
| Cultivation | 6.0 | 32.1 | 151.6 | 4.72 |
| pH | 6.5 | 38.2 | 204.6 | 5.36 |
| | 7.0 | 42.1 | 224.8 | 5.33 |
| | 7.5 | 40.4 | 188.4 | 4.66 |
| | 8.0 | 49.7 | 110.6 | 2.23 |
| Stirring | 300 | 36.4 | 180.2 | 4.95 |
| (rpm) | 400 | 40.2 | 219.6 | 5.46 |
| | 500 | 44.6 | 235.8 | 5.29 |
| | 600 | 45.7 | 204.6 | 4.48 |
| Aeration | 0.5 | 34.2 | 204.6 | 5.98 |
| (vvm) | 1.0 | 45.4 | 250.4 | 5.51 |
| | 1.5 | 45.8 | 235.8 | 5.15 |
| | 2.0 | 43.7 | 219.8 | 5.03 |

### EXAMPLE 10

### Control of Dissolved Oxygen

Under the basic culture condition performed in Example 8, dissolved oxygen concentration in culture medium declined to about 0 after 24 hours of culture. When dissolved oxygen concentration was adjusted to 0˜10, 10˜20 or 20˜30% by controlling agitation. 0˜10% of dissolved oxygen concentration leaded to the best growth of biomass and biosynthesis of coenzyme Q₁₀. According to this experiment, the biomass quantity increased to 54.1 g/L and the amount of biosynthesized coenzyme Q₁₀ increased to 281.6 mg/L accordingly. Table 6 shows production of coenzyme Q₁₀ by BNQ-pGPRX11 according to the Dissolved oxygen concentration

TABLE 6
Production of coenzyme Q₁₀ by BNQ-pGPRX11 according to the
Dissolved oxygen concentration

| | Biomass | CoQ₁₀ | CoQ₁₀ |
|---|---|---|---|
| | (g/L) | (mg/L) | (mg/g-DCW) |
| No control | 44.9 | 250.4 | 5.57 |
| DO 0˜10% | 51.2 | 280.2 | 5.47 |
| DO 10˜20% | 48.1 | 265.8 | 5.52 |
| DO 20˜30% | 40.0 | 221.3 | 5.53 |

### EXAMPLE 11

### Fed-Batch Culture

For increasing the biomass quantity, fed-batch culture was applied. Therefore, 50 g/L of sugar was intermittently added upon exhausting the carbon source. According to this experiment, the biomass quantity was 70.2 g/L, the amount of biosynthesized coenzyme Q₁₀ was 352.6 mg/L and the amount of coenzyme Q₁₀ per biomass was 5.02 mg/g-cell. Table 7 shows the coenzyme Q₁₀ productivity according to fed-batch culture in comparison to batch culture.

TABLE 7
The coenzyme Q₁₀ productivity according to fed-batch culture in
comparison to batch culture

| | Cell mass | CoQ₁₀ | CoQ₁₀ | Productivity |
|---|---|---|---|---|
| | (g/L) | (mg/L) | (mg/g-DCW) | (mg/g-DCW) |
| Batch | 51.2 | 280.2 | 5.47 | 3.89 |
| culture | | | | |
| Fed-batch | 70.2 | 352.6 | 5.02 | 3.67 |
| culture | | | | |

### EXAMPLE 12

### The Optimal Concentration of Corn Steep Powder in Medium

Optimal concentration of corn steep powder used as a nitrogen source in the medium was measured according to the experiment. The experimental results revealed that the amount of biomass was 71.2 g/L; the amount of biosynthesized coenzyme Q₁₀ was 438.6 mg/L; and the amount of coenzyme Q₁₀ per biomass was 6.16 mg/g-biomass when 20 g/L of corn steep powder was added. Table 8 shows the amount of biomass, amount of biosynthesized coenzyme Q₁₀ and amount of coenzyme Q₁₀ per biomass according to the concentration of corn steep powder.

TABLE 8
The amount of biomass, amount of biosynthesized coenzyme Q₁₀ and
amount of coenzyme Q₁₀ per biomass according to the concentration of
corn steep powder

| | biomass | CoQ₁₀ | CoQ₁₀/biomass |
|---|---|---|---|
| | (g/L) | (mg/L) | (mg/g-DCW) |
| CSP 30 g/L | 62.4 | 361.0 | 5.78 |
| CSP 40 g/L | 71.2 | 438.6 | 6.16 |
| CSP 50 g/L | 72.6 | 403.7 | 5.56 |
| CSP 60 g/L | 70.2 | 352.6 | 5.02 |

### EXAMPLE 13

### The Optimal Concentration of Potassium Phosphate Monobasic and Potassium Diphosphate in Medium

Optimal concentration of potassium phosphate monobasic and potassium diphosphate were measured according to the experiment. It was confirmed that the optimal concentration was achieved when 1.6 g/L of potassium phosphate monobasic and potassium diphosphate were respectively added. According to the experiment, the amount of biomass was 71.4 g/L; the amount of biosynthesized coenzyme Q₁₀ was 472.6 mg/L; and the amount of coenzyme Q₁₀ per biomass was 6.62 mg/g-cell.

### EXAMPLE 14

### The Optimal Concentration of Ammonium Sulfate in Medium

According to the experiment, the optimal concentration of ammonium sulfate for producing coenzyme Q₁₀ in biomass was achieved, when 15 g/L of ammonium sulfate was added. After cultivation for 96 hours, the amount of biomass was 79.2 g/L; the amount of biosynthesized coenzyme Q₁₀ was 548.2 mg/L; and the amount of coenzyme Q₁₀ per biomass was 6.92 mg/g-cell. Table 9 show the amount of biomass, amount of biosynthesized coenzyme Q₁₀ and amount of coenzyme Q₁₀ per biomass according to the concentration sulfate.

TABLE 9
The amount of biomass, amount of biosynthesized coenzyme Q₁₀ and
amount of coenzyme Q₁₀ per biomass according to the concentration of
ammonium sulfate

| Ammonium | biomass | CoQ₁₀ | CoQ₁₀ |
|---|---|---|---|
| sulfate | (g/L) | (mg/L) | (mg/g-DCW) |
| 5 g/L | 71.4 | 472.6 | 6.62 |
| 10 g/L | 78.1 | 521.7 | 6.68 |
| 15 g/L | 79.2 | 548.2 | 6.92 |
| 20 g/L | 79.0 | 500.1 | 6.33 |

### EXAMPLE 15

### Fed-Batch Culture Using pH-Stat

The fed-batch culture for carbon source using pH-stat and the conventional fed-batch culture intermittently feeding carbon source were carried out. Above two fed-batch culture methods were compared so as to find the best mode for increasing the amount of biomass and the amount of coenzyme Q₁₀. The experimental results showed that fed-batch culture using pH-stat was better efficient than fed-batch culture by intermittent feeding. According to this experiment, the amount of biomass was 88.2 g/L; the amount of bioosynthesized coenzyme Q₁₀ was 642.1 mg/L; the amount of coenzyme Q₁₀ per biomass was 7.30 mg/g-cell; and the productivity was 6.69 mg/g-hr.

1. An isolated 1-deoxy-D-xylulose 5-phosphate synthase (DXS) gene of SEQ ID NO: 1 from Agrabacterium tumefaciens.

2. A 1-deoxy-D-xylulose 5-phosphate synthase (DXS) of SEQ ID NO: 2.

3. A recombinant expression vector (pGPRX11) inserted with both decaprenyl diphosphate (DPS) gene and 1-deoxy-D-xylulose 5-phosphate synthase (DXS) gene.

4. A transformed Agrabacterium tumefaciens BNQ-pGPRX11 (Accession No. KCCM-10554) harboring a recombinant expression vector (pGPRX11).

5. A fermentation method for maximum production of coenzyme Q₁₀ using a transformed Agrabacterium tumefaciens deposited to Korean Culture Center of Microorganism with accession number KCCM-10554 comprising the steps of:
i) fermenting transformed cells on production medium comprising 30˜50 g/L of corn steep powder, 0.3˜0.7 g/L of KH₂PO₄, 0.3˜0.7 g/L of K₂HPO₄, 12˜18 g/L of ammonium sulfate, 1.5˜2.5 g/L of lactic acid, 0.2˜0.3 g/L of magnesium sulfate on condition that aeration rate of the medium is 0.8˜1.2 volume of air per volume of medium per minute, temperature 30˜34° C. and pH is 6.0˜8.0;
ii) removing the transformed cells and other residue from the fermentation medium; and
iii) separating and recovering coenzyme Q₁₀ from the fermentation medium of step (ii).


SEQUENCE LISTING (18 sequences)

SEQ ID NO: 1 - DNA, 1920 bp, Agrobacterium tumefaciens
ttgaccggaatgccacagaccccattgcttgaccgggtgaattttccatccgatctcaaggagatcgacgatcgcgacctgccggaactggcaagggagttgcgcgacgagatgatcgacgcggtgtcgagcaccggcgggcatctgggtgccggtctgggcgtggtggagctgacgattgccatccataaggtgttcaacacgccggaagaccggttgatcttcgatgtcggccaccaatgttatccgcacaagatcctgaccggccgtcgcgagcgcatccgcacgctgcgccaggaaggcggcctttccggtttcacccggcgggccgaaagcgaatatgacgatttcggcgccggccattcctccacctccatttccgccggtctcggcatggcggtggcagcgggtctcgacgaaagcgaccgcaaggtgatcgccgtcatcggcgacggctccatgtctgccggcatggcgttcgaggcgctcaacaatgcaggcgcgctcgacgcgcggctgatcgtcatcctcaacgacaacgacatgtcgattgcaccgccgacgggcgcgatgagcgcttatctcgcccgccttgcctccggccgcacctatatgggcttccgtgatttcggcaagaagctcacagcctatctcggcaagaccatcgaccgcgccattacccgcgccgtgacccatgcgcgtggttatgtgaccggcggtacgctgttcgaggagctcggtttctatcacatcggcccgatcgacggccattccttcgaccacctgctaccggtgctgcgcaatgtgcgcgacaaccagaaaggccctgtcctcatccatgtggtgacgcagaaaggcaagggttatgcgccggcggaagcggctgcggacaaatatcacggcgtcaacaagttcgacgtcatcaccggcgcacaggcgaaagccaaaccgaatgcgccgagctataccagcgtctttgccgaagcgctgatccaggaagcgaccctcgacgacaagatcgtcggcgtcaccgccgccatgccgaatggtaccggcctcgacaagatggccgagcttttcccgtcccgcaccttcgatgtcggaattgccgaacagcacgctgtcaccttcgcggcggggcttgcggctgacggctacaagccgttctgcgcgctttattccaccttcctgcagcgtggttacgaccagctggtgcatgacgtggcgatccagagcctgcccgtacgtttcccgatcgaccgcgccgggttcgtcggcgctgacggaccgacccatgccggctccttcgacaccactttcctcgccaccctgcccggtatggtggtgatggcggcggccgacgaggccgaactgaaacatatggtacgcacggcagcggcctatgatgaaggcccgatttccttccgttacccgcgcggcgaaggtgtgggcgtcgagatgcctgcacgcggtgaaatccttgagatcggcaagggccgcatcatcaaggaaggcaccaaggttgcgctgctctccttcggtacccggcttgcggaatgccttgcagccgccgaagatcttgatgccgccggactttcgacgaccgttgccgatgcgcgctttgccaagccgctcgatctcgatctcatccgccagcttgcggcccatcacgaagtgctggtgacgatcgaggaaggctcagtcggcggttttggcgcgcatgtgctgcatttcatggcaagcgccggattgctcgaccacggccccaaggtccggacgctgaccctgcccgaccagtgggtggagcaggccaagcccgagaccatgtatgccaatgccggcctcgaccgggccggcatcgtgtccaccgtgttcaatgcgctcggccagcgtcaggccggcgtcggtttcgccggctga

SEQ ID NO: 2 - protein, 639 aa, Agrobacterium tumefaciens
LTGMPQTPLLDRVNFPSDLKEIDDRDLPELARELRDEMIDAVSSTGGHLGAGLGVVELTIAIHKVFNTPEDRLIFDVGHQCYPHKILTGRRERIRTLRQEGGLSGFTRRAESEYDDFGAGHSSTSISAGLGMAVAAGLDESDRKVIAVIGDGSMSAGMAFEALNNAGALDARLIVILNDNDMSIAPPTGAMSAYLARLASGRTYMGFRDFGKKLTAYLGKTIDRAITRAVTHARGYVTGGTLFEELGFYHIGPIDGHSFDHLLPVLRNVRDNQKGPVLIHVVTQKGKGYAPAEAAADKYHGVNKFDVITGAQAKAKPNAPSYTSVFAEALIQEATLDDKIVGVTAAMPNGTGLDKMAELFPSRTFDVGIAEQHAVTFAAGLAADGYKPFCALYSTFLQRGYDQLVHDVAIQSLPVRFPIDRAGFVGADGPTHAGSFDTTFLATLPGMVVMAAADEAELKHMVRTAAAYDEGPISFRYPRGEGVGVEMPARGEILEIGKGRIIKEGTKVALLSFGTRLAECLAAAEDLDAAGLSTTVADARFAKPLDLDLIRQLAAHHEVLVTIEEGSVGGFGAHVLHFMASAGLLDHGPKVRTLTLPDQWVEQAKPETMYANAGLDRAGIVSTVFNALGQRQAGVGFAG

SEQ ID NO: 3 - DNA, 27 bp, Artificial Sequence
  primer
catcaaagatcctcctcgaccggccgc

SEQ ID NO: 4 - DNA, 24 bp, Artificial Sequence
  primer
cgcagtgctgtctccggcgatgcc

SEQ ID NO: 5 - DNA, 21 bp, Artificial Sequence
  primer
ctcggccatcttgtcgaggcc

SEQ ID NO: 6 - DNA, 21 bp, Artificial Sequence
  primer
attcggcatggcggcggtgac

SEQ ID NO: 7 - DNA, 21 bp, Artificial Sequence
  primer
gccgacgatcttgtcgtcgag

SEQ ID NO: 8 - DNA, 19 bp, Artificial Sequence
  primer
gcagctttcggtcgccaag

SEQ ID NO: 9 - DNA, 26 bp, Artificial Sequence
  primer
ggatccttgaccggaatgccacagac

SEQ ID NO: 10 - DNA, 25 bp, Artificial Sequence
  primer
aagcttctcagccggcgaaaccgac

SEQ ID NO: 11 - DNA, 25 bp, Artificial Sequence
  primer
aagcttttgccgcgcaaggcgtcag

SEQ ID NO: 12 - DNA, 28 bp, Artificial Sequence
  primer
acgcgttcagttgagacgctcgatgcag

SEQ ID NO: 13 - DNA, 26 bp, Artificial Sequence
  primer
aagcttttgaccggaatgccacagac

SEQ ID NO: 14 - DNA, 24 bp, Artificial Sequence
  primer
gaattctcagccggcgaaaccgac

SEQ ID NO: 15 - DNA, 29 bp, Artificial Sequence
  primer
ctcgaggaagttcatttcatttggagagg

SEQ ID NO: 16 - DNA, 24 bp, Artificial Sequence
  primer
atcgattcagccggcgaaaccgac

SEQ ID NO: 17 - DNA, 19 bp, Artificial Sequence
  primer
atccttcgcaagacccttc

SEQ ID NO: 18 - DNA, 19 bp, Artificial Sequence
  primer
gcttagctcatcgcagatc